(19) United States
(12) Patent Application Publication
(10) Pub. No.: US 2022/0194816 A1
(43) Pub. Date: Jun. 23, 2022

(54) APPARATUS AND METHOD FOR PROVIDING PURIFIED WATER

(71) Applicant: VWS (UK) LIMITED, High Wycombe (GB)

(72) Inventors: Nigel EMERY, Monks Risborough Buckinghamshire (GB); Lee UNDERWOOD, High Wycombe Buckinghamshire (GB); Paul Matthew NGUI, Brentwood Greater London (GB)

(21) Appl. No.: 17/599,660

(22) PCT Filed: Mar. 27, 2020
(86) PCT No.: PCT/GB2020/050825

§ 371 Date: Sep. 29, 2021

(30) Foreign Application Priority Data
Apr. 3, 2019 (GB) 1904673.9

(51) Int. Cl. C02F 1/00; C02F 9/00; G01N 33/18; G01N 27/08

(52) U.S. Cl. CPC C02F 1/008; C02F 9/005; C02F 1/006; G01N 33/1826; C02F 2103/04; C02F 2301/046; C02F 2209/05; C02F 2209/44; C02F 2209/40; G01N 27/08

(57) ABSTRACT

A method of providing a dispense purified water stream from a water purification apparatus involving passing a water inlet stream through a first water purification station to provide a first internal purified water stream, passing the first internal purified water stream to an internal reservoir, and providing a second internal purified water stream from the reservoir, passing the second internal purified water stream into a recirculation loop, measuring the conductivity of the second internal purified water stream; passing the second internal purified water stream to a second water purification station to provide a third internal purified water stream passing the recirculated water return stream into the internal reservoir; calculating the purity of the first internal purified water stream using the measurement of the conductivity of the second internal purified water stream.

The present invention relates to an apparatus and method for providing purified water, in particular a water purification apparatus that has at least two stages of purification, the second stage of purification being in a recirculation loop and having determination of the conductivity of the water in the recirculation loop that is used to also determine the conductivity of the water output from the first purification stage.

Water purification apparatus and units for use in laboratories and healthcare facilities are well known. Generally, they involve the reduction and/or removal of contaminants and impurities to very low levels. They typically contain a variety of technologies that remove particles, colloids, bacteria, ionic species and organic substances and/or molecules to a specified purity.

A typical water purification apparatus will have an inlet to provide water to a first purification stage that provides partially purified water into a reservoir. A recirculation loop from the reservoir passes through a second purification stage with the water exiting the second purification stage either being taken from the water purification apparatus as a product water, possibly through a third purification stage at the point of dispense, or the water exiting the second purification stage is returned to the reservoir. The recirculation of the water helps to maintain the high level of purity required.

It is preferable to know the purity or quality of the water after each purification stage to know how the purification stage is performing, and hence know if and when components in the purification stage are not optimally functioning, and may require maintenance or replacement.

Water purity or water quality is often determined by the conductivity of the ions dissolved in the water, and can be expressed as siemens/meter (S/m) or microsiemens/centimeter (μS/cm). Potable water typically has a conductivity of between 100 to 1000 μS/cm, and varies depending on its source. As the water is purified, the amount of ions decreases and the conductivity similarly decreases, until reaching a limit caused by the natural dissociation of water molecules into hydrogen and hydroxide ions of 0.055 μS/cm, as corrected to a standard temperature of 25° C. This conductivity may also be expressed by the inverse unit of resistivity such that the theoretical limit is 18.2 MΩ·cm or Mohm·cm.

As it is purified the conductivity of water may pass through a series of generic water purity standards so that it may typically be called:
type 3 below 20 μS/cm (0.05 MΩ·cm),
type 2 below 1 μS/cm (1 MΩ·cm), and
type 1 below 0.056 μS/cm (18 MΩ·cm)

All as corrected to a standard temperature of 25° C.

Conductivity measurement devices are known and typically involve the measurement of the conductance of the solution as it passes between two electrodes. However, conductivity measuring devices have an associated cost, both in their parts and in requiring an input to the control system of the water purification apparatus. They also have a weight and size, increasing the weight and size of the water purification apparatus.

These limitations mean that some water purification devices do not have conductivity measurement devices to determine the purity of the first purification stage.

It is an object of the present invention to provide a simple and cost-effective water purification apparatus and method for operating a water purification apparatus that uses a conductivity sensor in a recirculation loop to determine the conductivity of the water entering an associated reservoir.

Thus, according to one aspect of the present invention, there is provided a method of providing a dispense purified water stream, from a water purification apparatus, comprising at least the steps of:
(a) passing a water inlet stream through a first water purification station comprising one or more first water purification process units to provide a first internal purified water stream that is of higher purity than the water inlet stream;
(b) passing the first internal purified water stream to an internal reservoir within the water purification apparatus through a water inlet, the internal reservoir holding a volume of second purified water, and providing a second internal purified water stream from the reservoir;
(c) passing the second internal purified water stream from the reservoir into a recirculation loop;
(d) measuring the conductivity of the second internal purified water stream in the reservoir, or in the recirculation loop, or both;
(e) passing the second internal purified water stream to a second water purification station comprising one or more second water purification process units, and providing a third internal purified water stream that is of higher purity than the second internal purified water stream;
(f) providing by selecting from the third internal purified water stream, either a dispense purified water outlet stream, or a recirculated water return stream, or both;
(g) passing the recirculated water return stream into the internal reservoir from the recirculation loop;
(h) calculating the purity of the first internal purified water stream using the measurement of the conductivity of the second internal purified water stream.

Optionally, the method further includes the step of measuring the conductivity of the third internal purified water stream.

Optionally, the conductivity of the second internal purified water stream is measured in the reservoir.

Optionally, the conductivity of the second internal purified water stream is measured in the recirculation loop.

Optionally, the conductivity of the second internal purified water stream is measured in both the reservoir and the recirculation loop.

Optionally, the method further includes the step of:
measuring a time period between reaching a predetermined volume of second purified water in the internal reservoir from first internal purified water stream entering the internal reservoir, and a predetermined conductivity value of the second internal purified water stream.

Optionally in such method, the predetermined volume of second purified water is when the internal reservoir is wholly or substantially full.

Optionally in such method, the predetermined conductivity method is a nominal value such as 0.5 μS/cm. 1 μS/cm, 2 μS/cm, etc.

Optionally, the method further includes the step of: measuring conductivity of the second internal purified water stream over time. Optionally, over time until the measurement of conductivity of the second internal purified water stream reaches a predetermined value such as listed above, optionally a relatively constant value or steady state.

Optionally, the method further includes the steps of:
measuring the rate of fill of the internal reservoir by the first internal purified water stream, and
using the measurement rate of fill in calculating the purity of the first internal purified water stream.

Optionally, the method further includes the steps of:
measuring the rate of flow of water in the recirculation loop; and
using the measurement in calculating the purity of the first internal purified water stream.

Optionally, the method of the present invention includes two or more of the above further steps, whose combined measurements can be used to calculate the purity of the first internal purified water stream,

Optionally, the measurement of the conductivity of the second internal purified water stream is carried out by a first conductivity measurement device.

Optionally, any measurement of the conductivity of the third internal purified water stream is carried out by a second conductivity measurement device.

Preferably the water is passed around the recirculation loop by the action of a pump, preferably a positive displacement pump.

Preferably, one or more of the first and/or second water purification process units in the first and second water purification stations includes one or more of the following group comprising: an oxidiser, a deioniser. Such items are discussed in more detail herein.

Preferably, the one or more first water purification process units in the first water purification station includes one or more of the following group comprising: a reverse osmosis unit, a capacitive deionisation unit. Such items are discussed in more detail herein.

Preferably, the one or more second water purification process units in the second water purification station includes one or more of the following group comprising: ion exchange resin, electrodeionisation. Such items are discussed in more detail herein.

Optionally the one or more second water purification process units in the second water purification station further includes one or more of the following group comprising: UV oxidation, ozonation, electrochemical oxidation, ultrasonic oxidation. Such items are discussed in more detail herein.

Optionally, at least one of or each of the first, second and third dispense purified water outlet streams is pressurised or pumped. Preferably a valve is located after the pump such that the second dispense purified water stream is a pressurised flow.

Preferably, the dispense purified water stream is of ultra-pure water of resistivity>15 MΩ·cm, more preferably >18 MΩ·cm.

Operation of the water purification apparatus may be programmed or controlled by one or more control systems, typically using one or more microprocessors preferably sited on one or more printed circuit boards (PCB), with subsequent operational control of valves and pump based on inputs from a user interface, such as a touchscreen and/or input buttons, and inputs from sensors such as level sensors, water quality measurement devices and where fitted flow measurement devices, as defined by software and firmware in the microprocessor.

Optionally, at least a second dispense purified water stream of second purified water is provided from the water purification apparatus, either from the reservoir or the recirculation loop, or both.

The skilled person recognises that the present invention is not intended to only provide a continuous dispense purified water outlet stream over time, and that in providing by selecting from the third internal purified water stream, either a dispense purified water outlet stream, or a recirculated water return stream, or both, that there will still be some portion of the third internal purified water stream becoming a recirculated water return stream over time to provide a flow back into the internal reservoir from the recirculation loop. That is, the present invention provides a dispense purified water outlet stream from the third internal purified water stream in the recirculation loop, and a recirculated water return stream continues to pass into the internal reservoir from the recirculation loop when providing a dispense purified water outlet stream is not fully selected from the third internal purified water stream. The normal mode of operation will be with recirculation, with intermittent times of dispense (of the dispense purified water outlet stream) mode.

Optionally, a third dispense purified water stream of first purified water is provided from the water purification apparatus from the first internal purified water stream.

The water purification apparatus may be constructed within a single housing containing at least the first and second water purification stations, the internal reservoir, a pump, and first and second conductivity measurement devices: optionally all of the purification technologies, reservoir, pumps, valves and controls.

Optionally, the water purification apparatus is portable, for example by one person around a laboratory requiring connection to feedwater and electricity at any particular location.

Preferably, the water purification apparatus has a mass of <15 kg when the reservoir is empty of water.

Preferably the water purification apparatus has a mass of <22 kg when the reservoir is full of water.

Optionally the water purification system has connections to attach a remote dispense point or to extend the recirculation loop external to the housing such as to a piece of equipment requiring the purified water.

In one embodiment, the present invention is a water purification apparatus including one or more water connections able to extend the dispense purified water stream or the recirculation loop beyond or outside the housing.

Preferably the water purification apparatus includes a first water quality measurement device to measure the water quality of the second purified water in the internal reservoir, a second water quality measurement device to measure the water quality of the third internal purified water stream, and a reservoir level sensor that can measure the amount of water in the internal reservoir.

Preferably the water purification apparatus includes means to measure the amount of water in the reservoir. This may be by one or more level sensors as known in the art, for example to measure the position, height or pressure of the water. It may additionally or alternatively involve the measurement of the amount of water flowing into and out of the reservoir.

Optionally, the water purification apparatus further comprises a grey water outlet stream from the first water purification station.

According to another aspect of the present invention, there is provided a water purification apparatus able to provide a dispense purified water stream, that has at least two stages of purification and a reservoir thereinbetween, the second stage of purification being in a recirculation loop and having conductivity determination in the recirculation loop or in the reservoir or both that is able to determine the conductivity of the water output from the first purification stage.

Optionally, the water purification apparatus includes one or more of the embodiments as described herein.

Optionally, the water purification apparatus of the present invention comprises;
a water inlet;
a first water purification station comprising one or more first water purification process units to provide a first internal purified water stream that is of higher purity than the water inlet stream;
an internal reservoir able to hold a volume of second purified water comprising at least the first internal purified water stream, and able to provide a second internal purified water stream from the reservoir;
a recirculation loop for the second internal purified water stream from the reservoir into a recirculation loop;
a first conductivity measurement device able to measure the conductivity of the second internal purified water stream,
a second water purification station comprising one or more second water purification process units, able to receive the second internal purified water stream, and able to provide a third internal purified water stream that is of higher purity than the second internal purified water stream;
a valve able to select from the third internal purified water stream either a dispense purified water outlet stream or a recirculated water return stream or both;
a passage to pass the recirculated water return stream into the internal reservoir from the recirculation loop;
a calculator able to calculate the purity of the first internal purified water stream using the measurement of the conductivity of the second internal purified water stream.

Optionally, the water apparatus further comprises a second conductivity measurement device to measure the conductivity of the third internal purified water stream.

In one embodiment of the present invention, there is provided a water purification apparatus able to provide at least three dispense purified water streams of different water purities from the water purification apparatus, comprising at least:

(i) a water inlet stream;

(ii) a first water purification station comprising one or more first water purification process units connected to the water inlet and able to provide a first internal purified water stream;

(iii) a first valve able to select from the first internal purified water stream either a first dispense purified water stream or a first continuing water stream or both;

(iv) an internal reservoir within the water purification apparatus adapted to receive the first continuing water stream through a water inlet, to hold a volume of second purified water, and to provide a second internal purified water stream,

(v) a second valve able to dispense a second dispense purified water stream from the water purification apparatus;

(vi) a second water purification station comprising one or more second water purification process units able to receive the second internal purified water as a second continuing water stream and able to provide a third internal purified water stream;

(vii) a third valve able to select from the third internal purified water stream either a third dispense purified water stream or a third continuing water stream or both;

(viii) a recirculation loop able to return the third continuing water stream into the internal reservoir; and

(ix) a pump able to pump the second internal purified water stream from the internal reservoir around the recirculation loop.

Optionally, the water apparatus consists of or consists essentially of:
a water inlet;
a first water purification station comprising one or more first water purification process units to provide a first internal purified water stream that is of higher purity than the water inlet stream;
an internal reservoir able to hold a volume of second purified water comprising at least the first internal purified water stream, and able to provide a second internal purified water stream from the reservoir;
a recirculation loop for the second internal purified water stream from the reservoir into a recirculation loop;
a first conductivity measurement device able to measure the conductivity of the second internal purified water stream,
a second water purification station comprising one or more second water purification process units, able to receive the second internal purified water stream, and able to provide a third internal purified water stream that is of higher purity than the second internal purified water stream;
a valve able to select from the third internal purified water stream either a dispense purified water outlet stream or a recirculated water return stream or both;
a passage to pass the recirculated water return stream into the internal reservoir from the recirculation loop;
a calculator able to calculate the purity of the first internal purified water stream using the measurement of the conductivity of the second internal purified water stream.

Optionally, the water apparatus further consists of or consists essentially of a second conductivity measurement device to measure the conductivity of the third internal purified water stream.

According to another aspect of the present invention, there is provided use of a measurement of the conductivity of a second internal purified water stream in a water purification apparatus able to provide a dispense purified water stream, and having at least two stages of purification, the second stage of purification being in a recirculation loop and having a recirculation loop, the measurement being able to determine the conductivity of the water output from the first purification stage.

Ions dissolved in water result in the water having a conductivity that is used as a measure of its purity. Potable water typically has a conductivity of between 100 to 1000 μS/cm and varies depending on its source.

The skilled man is aware of the relationship between conductivity and resistivity, such that either one or both measurements can be made by a suitable measurer or meter. Thus, the term “conductivity value” as used herein relates to the measurement of the conductivity and/or resistivity of a water stream. The skilled man is also aware that conductivity and/or resistivity measurements or values are temperature dependent. Commonly, a temperature of 25° C. is used as a standard temperature when discussing and comparing conductivity and/or resistivity measurements, such that the conductivity of “pure” water is considered to be 0.055 μS/cm and the resistivity is considered to be 18.2 MΩ-cm, at 25° C.

There are many water quality standards published throughout the world with water purity requirements that are expressed, at least, by the resistivity of the water at a specific temperature, usually 25° C. such that requirements can be specified as, from most pure to least pure of 18.2 MΩ·cm, >18 MΩ·cm, >10 MΩ·cm, >5 MΩ·cm, >1 MΩ·cm or >0.05 MΩ·cm. Other specifications on the water purity may be defined by the water's level of organic, microbial or endotoxin content. The purest of these purity levels are often referred to as ‘ultra-pure’ or ‘ultra-purified’ water while the less pure are more generally referred to as ‘pure’ or ‘purified’ water.

As the water is purified its conductivity decreases and its resistivity correspondingly increases.

Conductivity measurement can be determined as found in ASTM International D1125 Standard Test Methods for Electrical Conductivity and Resistivity of Water.

By analysing the conductivity of the second internal purified water stream, the present invention can determine the conductivity of the first internal purified water stream entering the reservoir. This allows or helps to determine the state of the components in the first water purification station, and allows or helps to identify when components require changing, or identifying if there are issues with the content of the water inlet stream.

As the first internal purified water stream enters the reservoir the conductivity of the second purified water in the reservoir increases. However the action of recirculating the second purified water around the recirculation loop, and its purification in the second water purification station, results in its return to the reservoir as a recirculated water return stream with all or a majority of the remaining ions removed. This limits the increase in conductivity of the water in the reservoir, and a steady level of the conductivity of the second purified water can be achieved when the amount of ions entering the reservoir in the first purified water stream equals the amount of ions being removed during the (re)circulation through the second water purification station. It is therefore possible to measure the conductivity of the second purified water either in the reservoir, or in the recirculation loop before the second water purification station, or both to determine the conductivity of the first internal purified water stream, using suitable computation such as an algorithm or a look up table.

Additionally or alternatively, once the reservoir is full and no further first internal purified water enters the reservoir, then the recirculation will purify the second purified water in the reservoir by the action of its being passed around the recirculation loop through the second water purification station. By also measuring the time taken to achieve a specific water conductivity, the conductivity of the first internal purified water stream can also or further be determined using suitable computation such as an algorithm or a look up table.

The conductivity of the second purified water in the reservoir and entering the recirculation loop may also be affected by the rate of flow of the first internal purified water entering the reservoir. If this rate of flow is a constant value, or near to a constant value, then this value can be used to help determine the conductivity of the first internal purified water stream, for example from an algorithm or look up table. If this rate of flow varies, then monitoring the rate of change of the level in the reservoir, by means known in the art, can determine the rate of flow of the first internal purified water. The rate of flow of the first internal purified water can then be used in the algorithm to help determine the conductivity of the first inlet purified water, or a modification can be made to the outcome, from the look up table based on the rate of flow.

The rate of flow in the recirculation loop may also affect the conductivity of the second purified water during filling of the reservoir, and the time taken to purify to a specific water conductivity once the reservoir is full. If this rate of flow is a constant value, or near to a constant value, which may be achieved for example by the use of a positive displacement pump to recirculate the water around the recirculation loop, its value can also be used to help determine the conductivity of the first internal purified water stream from the algorithm or look up table. If there is variation in the rate of flow around the recirculation loop, then a flow measurement device may be used in the recirculation loop, and the value from the flow measurement device can be also used to help determine the conductivity of the first inlet purified water, or be used to modify the outcome from the look up table, etc.

Other parameters that may be of importance in the purified water, such as the total organic contamination (TOC) being less than 500 ppb, potentially <5 ppb, or having a bacterial contamination of less than 100 cfu/ml, potentially <1 cfu/ml.

According to one embodiment of the present invention, recirculation around the recirculation loop is wholly or substantially continuous. Such active use may be during a laboratory ‘working hours’, and as long as there is enough water in the internal reservoir. When the level in the reservoir is too low, as indicated for example by a level control, then the pump could be turned off to prevent wear on the pump.

When the method of the present invention is not continuously or regularly required, for example outside working or operational hours of a laboratory, the water purification system would typically only recirculate water from the reservoir intermittently, say 5 minutes per hour. This would maintain a high level of purity in the reservoir while reducing wear on any electrical components such as the pump motor or oxidisers such as ultraviolet light devices, and hence increase their life.

The first water purification station preferably includes one process unit being a deioniser to purify the inlet or feed water to the first dispense purified water quality desired. Preferably the deioniser is a reverse osmosis unit or a capacitive deionisation unit. Operation of these units are known in the art and not described in detail herein. Waste ions can be passed from the first water purification station as a ‘grey water’ through a suitable outlet, that may be used for general purposes in the laboratory where water purity is not of concern.

The one or more first water purification process units may also include a filter to remove particles, and/or activated carbon for the removal of chlorine or chloramines from the feed water that would damage process equipment such as reverse osmosis or capacitive deionisation membranes.

The one or more first water purification process units may further include ion exchange resin in the sodium form to soften the feed water, by removing calcium ions that may precipitate in downstream purification processes.

The one or more second water purification process units may include a deioniser to purify the second internal purified water to a higher or third purified water quality. Unlike any deioniser in the first water purification station, a deioniser in the second water purification station may be required to remove dissolved carbon dioxide from the water. Preferably a deioniser in the second water purification station is a cartridge of ion exchange resin or an electrodeionisation unit. Operation of these units are known in the art and not described in detail herein.

Additionally, the one or more second water purification process units may further include one or more units for processes for oxidation of the water passing therethrough, either for inactivation of micro-organisms or for oxidation of organic molecules or both.

One common oxidiser involves the use of ultraviolet light, and the ultraviolet treatment of water for decomposing organic compounds or substances in water is well known in the art. Generally, ultraviolet light is able to decompose many organic compounds and substances that are contained or are residues in water, by oxidising them to form ionic or ionisable species that may be removed in the deioniser. Apparatus and instruments for providing suitable ultraviolet light are well known in the art, and may include one or more LEDs and typically involve emitting ultraviolet light at one or more specific wavelengths in an area or space in which the water is held or through which the water passes.

Alternatively or additionally, the oxidiser is a chemical oxidising species, such as a peroxide or ozone, which may be added or electrically generated or generated electrochemically, optionally in the relevant water from oxygen dissolved within it. Such oxidising species act on organic molecules to break them down, and where the organic molecules are associated with viable species, render the species non-viable.

Alternatively or additionally, the oxidiser involves ultrasonics, which may be used either to directly break down the bonds in organic molecules, or to create oxidising species that then cause such breakages.

Additionally, the one or more second water purification process units may further include a size exclusion filter such as an ultrafilter or microfilter, or a charged filter.

In a further embodiment of the present invention the first water quality measurement device is also used to determine the total organic content (TOC) of the dispense purified water stream such that the first water quality measurement device is used to determine each of the conductivity of the first purified water stream, the conductivity of the second purified water stream and the TOC of the dispense purified water outlet stream.

The determination of the TOC of a water stream based on the change in conductivity value on passage through an oxidiser is well known in the art, and generally comprises measuring the conductivity and/or a related value of the water stream before and after the oxidiser and then using the change in conductivity to calculate the TOC in the water stream prior to the oxidiser.

The relationship between TOC and the conductivity generated is a function of the oxidising device's properties, its housing's geometry, the rate of flow and the concentration and the nature of the species in the water stream entering the oxidiser. The change in conductivity will also be a function of the conductivity of the water stream entering the oxidiser.

These effects can be determined experimentally for the actual components being used, and a calibration can be produced to provide a known or expected level of oxidation of organic substances during standard and/or normal operation of the oxidiser.

Typically, this provides a known or expected level of oxidation between 50 and 100%, such as 70% or 80%. The efficiency of an oxidiser may be estimated by periodically increasing the time the recirculated water stream spends in the oxidiser sufficiently to ensure complete oxidation of the or any organic substances present. The relationship between the change in conductivity during normal operation and the change in conductivity during complete oxidation can be used to check the efficiency of the oxidation and the values being used in the algorithms, and modify these values or alert the user, such as raising an alarm, as necessary.

Methods for improving the accuracy of determining the TOC are described in WO2010/043896A.

Embodiments of the present invention will now be described by way of example only and with reference to the accompanying drawings in which:

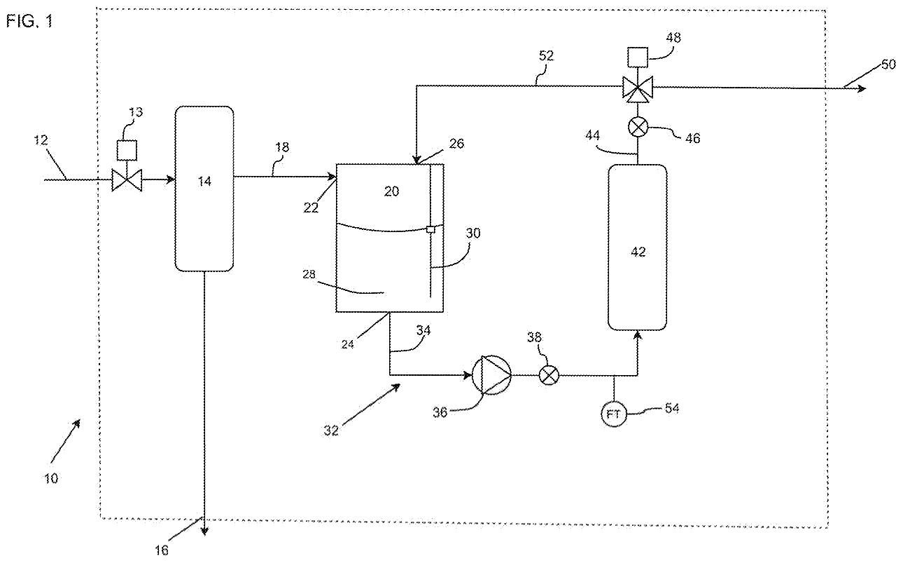

FIG. 1 is a simplified schematic view of a first water purification apparatus and method for providing purified water according to embodiments of the present invention; in particular a water purification apparatus that has at least two stages of purification, the second stage of purification being in a recirculation loop and having conductivity determination in the recirculation loop that is used to also determine the conductivity of the water output from the first purification stage;

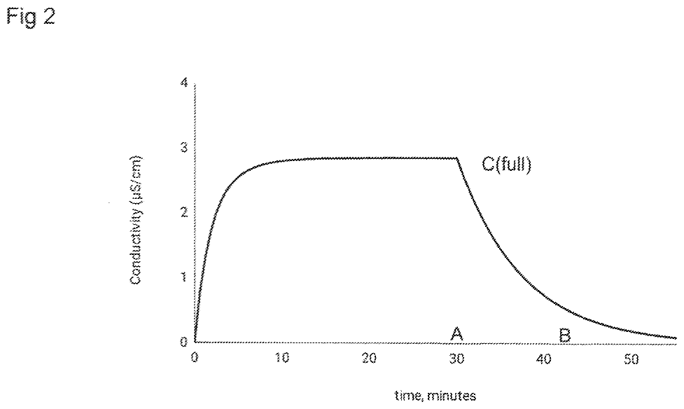

FIG. 2 is a chart detailing conductivity as measured by a first conductivity cell used to indirectly determine the conductivity of a first internal purified water stream involved in the present invention;

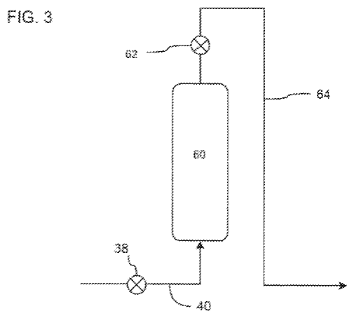

FIG. 3 is a schematic view of a section of a water purification apparatus for determining the total organic content of the purified water produced by the water purification apparatus; and

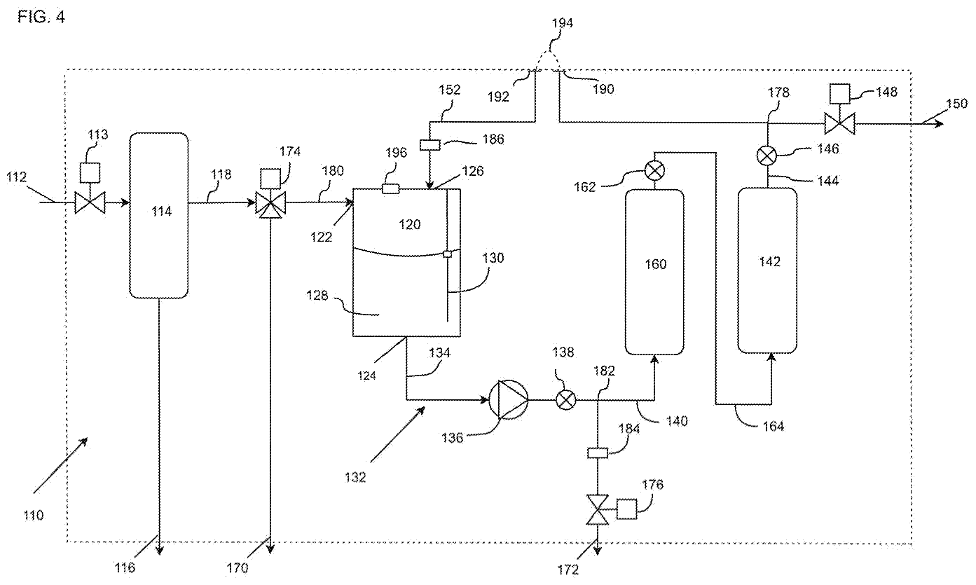

FIG. 4 is a schematic view of a second water purification apparatus and method according to a further embodiment of the present invention wherein an in-line conductivity measurement is used for determining the water conductivity at two different locations as well as determining the total organic content of one of the waters.

Referring to the drawings, FIG. 1 is a schematic diagram of one embodiment of the present invention. It shows a water purification apparatus 10 having an inlet for connection to a supply of potable or similar water from within a laboratory. The pipe for the water inlet stream 12 is connected to an inlet electrically activated valve 13, such as a solenoid valve, to control the flow of water into the apparatus 10. The outlet from the inlet solenoid valve 13 is connected to the first water purification station 14, able to purify the water inlet stream 12 to create a first purified water, which exits the first water purification station 14 as a first internal purified water stream 18.

The first water purification station 14 contains one or more deionising technologies, such as reverse osmosis or capacitive deionisation, to achieve the purification, and a grey water outlet 16 able to provide a water stream that may be used for general purposes in the laboratory where water purity is not of concern.

The first water purification station 14 may also contain other technologies able to filter the inlet water stream 12, to remove particles prior to the deionising technology.

The first water purification station 14 may further contain technologies such as activated carbon, to remove chlorine or chloramines from the inlet water stream 12 prior to the deionising technology.

The first water purification station 14 may further contain technologies such as ion exchange resin to soften the inlet water stream 12 by exchanging divalent ions for sodium ions after the deionising technology.

The skilled man can see that the first water purification station 14 may include one or more water purification process units able to provide one or more of the above technologies, and generally known in the art.

The first internal purified water stream 18 is passed to an internal reservoir 20 without passing through any conductivity measuring device.

The internal reservoir 20 can be any suitable shape and design and volume. Optionally, the internal reservoir has a volume in the range of 3 to 10 litres, and has a first water inlet 22 for the first internal purified water stream 18. The internal reservoir 20 also has an outlet 24 for water to exit into a recirculation loop 32, and a second inlet 26 for the returning recirculated water as described hereinafter.

The internal reservoir 20 also contains a level sensor 30 to determine the amount of water in the reservoir. This may be by any means such as measuring the water pressure, optical measurement, use of floats or any such method known in the art.

The internal reservoir 20 contains a second purified water 28, being a mixture of first internal purified water stream 18 that has entered by first water inlet 22, and water that has entered by the second water inlet 26 which is more purified than the first internal purified water stream 18 as described hereinafter.

The second purified water 28 is drawn from the internal reservoir 20 as a second internal purified water stream 34, and passed around a recirculation loop 32 by an in-line pump 36. The recirculation loop 32 contains a second water purification station 42.

The first water purification apparatus 10 further includes a first in-line water quality measurement device 38 for measuring the conductivity of the second internal purified water stream 34 provided from the internal reservoir 20 and pump 36. The first water quality measurement device 38 may be a conductivity cell as known in the art, preferably with a cell constant of 0.02 or less.

The recirculation loop 32 may further contain a flowrate measuring device 54 to accurately determine the rate of flow of the water around the recirculation loop 32.

The second water purification station 42 contains one or more deionising technologies, such as ion exchange resin or electrodeionisation, able to remove ions and dissolved carbon dioxide from the water therein, to create a third purified water, which exits the second water purification station 42 as a third internal purified water stream 44. The second water purification station 42 may have a waste stream (not shown), that can return water containing ions removed from the second purified water to a point prior to the first water purification station 14, or pass the waste stream from the unit through the grey water outlet 16.

The second water purification station 42 may further contain oxidative technologies such as UV oxidation or ozone or peroxide production to remove viable bacterial contamination from the water.

The second water purification station 42 may further contain oxidative technologies such as UV, ozone, peroxide, sonolysis or electrochemical oxidation to break down organic molecules from the water.

The second water purification station 42 may further contain molecular filtration by size exclusion, such as microfiltration or ultrafiltration or by charged filters, to remove bacteria, molecules and particulate contamination from the water.

The third internal purified water stream 44 exiting the second water purification station 42 is passed through a second in-line water quality measurement device 46 for measuring the conductivity of the third internal purified water stream 44. The second water quality measurement device 46 may be a conductivity cell as known in the art, preferably with a cell constant of 0.02 or less.

The third internal purified water stream 44 is then passed to a to a dispense valve 48, optionally an electrically activated valve, such as a solenoid valve, from which it is either returned to the internal reservoir 20 through the second water inlet 26 as a recirculated water return stream 52, or some or all of the third internal purified water stream 44 may be passed from the water purification apparatus 10 as a dispense purified water outlet stream 50.

The first water purification apparatus 10 further includes a control system, not shown, such as a printed circuit board including a microprocessor. Readings from the first and second water quality measurement device 38, 46 are processed by the microprocessor and water purity is output to a user by display means as known in the art.

FIG. 2 shows a curve of how the conductivity of water measured at the first water quality measurement conductivity device 38 of the apparatus shown in FIG. 1, based on a volume in the recirculation loop 34 of 0.5 litres, and a recirculation flow rate of 1.0 l/min, will vary when 5 litres are taken from a 7 litre reservoir, and the reservoir is refilled back to 7 litres at a rate of 167 ml/min with a first purified water with a conductivity of 20 μS/cm.

At time=0, the internal reservoir 20 starts to fill with water from the first water inlet 22 and the conductivity of the second purified water 28 in the internal reservoir 20 increases to a conductivity approaching a steady level. At time A at 30 minutes, the reservoir becomes full, and a measurement of the conductivity “C(full)”, as measured by the first conductivity device 38, is taken. The microprocessor can then compare conductivity C(full) to a lookup table or use an algorithm to determine the conductivity of the first internal purified water stream 18 that has been fed into the reservoir 20.

For any particular equipment the volume of the reservoir and recirculation loop are fixed. The curve of conductivity approaches a steady level, presuming that the fill is for a long enough period. In the example described above, a fill of over 2.5 litres corresponding to 15 minutes is suitable.

Conductivity C(full) may be affected by changes in flow rate of the first purified water filling the reservoir 20, and/or of the rate of flow of the recirculated water return stream 52. Fill flow rate can be determined by monitoring the rate of change of level sensor 30. Variation in flow rate can then be added to the algorithm, or adjustment made to the lookup table.

It is preferable to use a positive displacement pump 36 for the recirculation loop 32 to provide a constant flow therein. Greater certainty of the flow can be achieved by the addition of a flow rate monitor 54 in the recirculation loop, and one may be desirable in the water purification system to provide a user with information regarding the amount of water the user is dispensing.

An additional or alternative method for determining the conductivity of the first purified water 18 is to measure the time taken to purify the second purified water 28 to a known conductivity. An example in FIG. 2 is the time taken for the second purified water 28 to reduce from C(full) to 0.5 μS/cm i.e. from t=A to t=B. Again a lookup table or algorithm can be used to determine the conductivity of the first purified water.

FIG. 3 shows a section of a water purification apparatus for determining the total organic content (TOC) of the dispense purified water outlet stream 50.

The first in-line water quality measurement device 38 is used to measure the conductivity of an inlet stream 40 to a photo-oxidation chamber 60 located either before, within or after the second water purification station 42. As the water passes through the chamber it is irradiated with UV light from one or more suitable UV irradiation devices, such as LEDs designed to emit specific wavelengths, or discharge tubes that emit some of their radiation at the required wavelengths. The radiation causes the bonds in the organic molecules to fracture creating smaller species typically ionic or ionisable species which can be removed in downstream processes prior to dispense. The ionic or ionisable species cause a decrease in the resistivity of the water passing through the photo-oxidation chamber. The photo-oxidation outlet stream 64 from the photo-oxidation chamber 60 passes through a third in-line water quality measurement device 62 for measuring the conductivity of the photo-oxidation outlet stream 64. From the measurement of the first in-line water quality measurement device 38 and third in-line water quality measurement device 62, and knowing the oxidative efficiency of the photo-oxidative chamber 60, the water purification apparatus control system can calculate the TOC of the dispense purified water outlet stream 50.

FIG. 4 shows a second embodiment of a water purification apparatus of the present invention. Apparatus may be used incorporating any combination of the components and features of FIG. 4 where they differ from those shown in FIG. 1.

The second water purification system 110 has the same or similar components and features of the first water purification apparatus 10 in FIG. 1 and the section for TOC determination in FIG. 3, and so uses some notation numbering of +100 to represent such similar components and features.

The second water purification apparatus 110 includes the photo-oxidation chamber 160 and third in-line water quality measurement device 162 from FIG. 3 in the recirculation loop 134 before the second water purification station 142 such that the ions and ionisable that are generated in the photo-oxidation chamber 160 are removed in the second water purification station 142 prior to the water being dispensed as the dispense purified water outlet stream 150 or returned to the reservoir 120.

The second water purification apparatus 110 further includes outputs from the water purification apparatus 110 of a first purified water outlet stream 170 and a second purified water outlet stream 172.

The first internal purified water stream 118 is passed to a first purified water valve 174, preferably an electrically actuated valve such as a solenoid valve, for selectively passing the first internal purified water stream, 118 to either a first purified water outlet as a first purified water outlet stream 170 from the water purification apparatus 110, or to the internal reservoir 120 as a first continuing water stream 180.

A first tee or tee-junction 182 in the recirculation loop 132, preferably located after the pump 136 (so that the second internal purified water stream 134 is under pressure or ‘pressurised’ relative to atmospheric pressure), allows the second internal purified water stream 134 to pass towards the photo-oxidation chamber 160 and second water purification station 142 or some of the second internal purified water stream 134 may also be passed via a flow limiter 184 and a second purified water valve 176, preferably an electrically operated valve, as a second dispense purified water stream 172 from the water purification apparatus 110.

The flow limiter 184 ensures that only part of the second internal purified water stream 134 exiting the pump 136 can be output as the second dispense purified water stream 172, and that the flow is maintained to the photo-oxidation chamber 160 and second water purification station unit 142.

In FIG. 4 an alternative way of controlling the dispense is shown to that in FIG. 1, and each may be used in either apparatus. In FIG. 4 the third internal purified water stream 144 is passed to a second tee 178 from where it may be passed through a 2-way dispense valve 148 to create the dispense purified water outlet stream 150, or returned to the reservoir 120 as the recirculated water stream 152, or both. The pressure for dispensing the outlet water is maintained within the recirculation loop 132 by a pressure sustaining device 186 known in the art. With this arrangement greater control of the rate of output of the dispense purified water outlet stream 150 can be achieved by the use of variable valves such as proportional or stepper motor valves.

The second water purification apparatus 110 further includes locations 190, 192 for connecting a remote dispenser, or for extending the recirculation loop 132 around a laboratory. If no remote dispenser or recirculation loop extension is required, then a link 194 is present.

The internal reservoir 120 also contains a composite vent filter 196 to allow air passage into and out of the reservoir 120, thus equilibrating the air pressure inside and outside the reservoir 120, while also preventing particles, bacteria or carbon dioxide to enter the reservoir 120.

1. A method of providing a dispense purified water stream, from a water purification apparatus, comprising at least the steps of:
(a) passing a water inlet stream through a first water purification station comprising one or more first water purification process units to provide a first internal purified water stream that is of higher purity than the water inlet stream,
(b) passing the first internal purified water stream to an internal reservoir within the water purification apparatus through a water inlet, the internal reservoir holding a volume of second purified water, and providing a second internal purified water stream from the reservoir,
(c) passing the second internal purified water stream from the reservoir into a recirculation loop,
(d) measuring the conductivity of the second internal purified water stream in the reservoir, or in the recirculation loop, or both;
(e) passing the second internal purified water stream to a second water purification station comprising one or more second water purification process units, and providing a third internal purified water stream that is of higher purity than the second internal purified water stream;
(f) providing by selecting from the third internal purified water stream, either a dispense purified water outlet stream, or a recirculated water return stream, or both;
(g) passing the recirculated water return stream into the internal reservoir from the recirculation loop;
(h) calculating the purity of the first internal purified water stream using the measurement of the conductivity of the second internal purified water stream.

2. The method as in claim 1 wherein the measurement of the conductivity of the second internal purified water stream is carried out by a first conductivity measurement device.

3. The method as in claim 1 further including the step of measuring the conductivity of the third internal purified water stream.

4. The method as in claim 3 wherein the measurement of the conductivity of the third internal purified water stream is carried out by a second conductivity measurement device.

5. The method as in claim 1 further including the step of:
measuring a time period between reaching a predetermined volume of second purified water in the internal reservoir from first internal purified water stream entering the internal reservoir, and a predetermined conductivity value of the second internal purified water stream.

6. The method as in claim 1 further including the steps of:
measuring the rate of fill of the internal reservoir by the first internal purified water stream, and
using the measurement rate of fill in calculating the purity of the first internal purified water stream.

7. The method as in claim 1 further including the steps of:
measuring the rate of flow of water in the recirculation loop; and
using the measurement in calculating the purity of the first internal purified water stream.

8. The method as in claim 1 wherein the dispense purified water outlet stream is ultra-pure water of resistivity>15 MΩ·cm.

9. The method as in claim 1 wherein the first and second water purification stations include at least a deioniser.

10. The method as in claim 9 wherein the deioniser in the first water purification station is selected from a group comprising: a reverse osmosis unit, a capacitive deionisation unit.

11. The method as in claim 9 wherein the deioniser in the second water purification station is selected from a group comprising: ion exchange resin, electrodeionisation.

12. The method as claimed in claim 1 wherein the second water purification station includes an oxidiser.

13. The method as in claim 12 wherein the oxidiser provides oxidation selected from a group comprising: UV oxidation, ozonation, electrochemical oxidation, ultrasonic oxidation.

14. The method as claimed in claim 1 further including the steps of:
passing the second internal purified water stream through a UV photo-oxidation chamber and oxidising organic molecules therein,
passing the stream from the UV photo-oxidation chamber through a third conductivity measuring device, and
calculating the total organic content of the dispense purified water outlet stream using the measurements from the first and third conductivity measuring devices.

15. A water purification apparatus able to provide a dispense purified water stream, that has at least two stages of purification and a reservoir thereinbetween, the second stage of purification being in a recirculation loop and having conductivity determination in the recirculation loop, or reservoir, or both, that is able to determine the conductivity of the water output from the first purification stage.

16. The water purification apparatus as claimed in claim 15 provided within a single housing containing the first and second water purification stations, the internal reservoir, a pump, and first and second water conductivity measurement devices.

17. The water purification apparatus as claimed in claim 15 further including one or more water connections able to extend the dispense purified water stream or the recirculation loop beyond or outside the housing.

18. The water purification apparatus as claimed in claim 15 being a portable water purification apparatus.

19. The water purification apparatus as claimed in claim 18 having a mass of <22 kg with a full reservoir of water.

20. The water purification apparatus as claimed in claim 18 having an empty mass of <15 kg.

21. The water purification apparatus as claimed in claim 15 comprising;
a water inlet for a water inlet stream;
a first water purification station comprising one or more first water purification process units to provide a first internal purified water stream that is of higher purity than the water inlet stream;
the reservoir able to hold a volume of second purified water comprising at least the first internal purified water stream, and able to provide a second internal purified water stream from the reservoir;
a recirculation loop for the second internal purified water stream from the reservoir into the recirculation loop;
a first conductivity measurement device able to measure the conductivity of the second internal purified water stream;
a second water purification station comprising one or more second water purification process units, able to receive the second internal purified water stream, and able to provide a third internal purified water stream that is of higher purity than the second internal purified water stream;
a valve able to select from the third internal purified water stream either a dispense purified water outlet stream or a recirculated water return stream, or both;
a passage to pass the recirculated water return stream into the internal reservoir from the recirculation loop;
a calculator able to calculate the purity of the first internal purified water stream using the measurement of the conductivity of the second internal purified water stream.

22. A water purification apparatus as claimed in claim 21 further comprising a second conductivity measurement device to measure the conductivity of the third internal purified water stream.
